(19) United States
(12) Patent Application Publication
(10) Pub. No.: US 2025/0170115 A1
(43) Pub. Date: May 29, 2025

(54) LENALIDOMIDE ORAL SOLUTION

(71) Applicant: A FINE HOUSE S.A., Koropi (GR)

(72) Inventor: Georgios LIOLIOS, Chalandri Attica (GR)

(73) Assignee: A FINE HOUSE S.A., Koropi (GR)

(21) Appl. No.: 18/725,045

(22) PCT Filed: Dec. 30, 2022
(86) PCT No.: PCT/EP2022/088100

§ 371 Date: Jun. 27, 2024

(30) Foreign Application Priority Data
Dec. 31, 2021 (EP) 21386084.4

(51) Int. Cl. A61K 31/454; A61K 9/00; A61K 9/08; A61K 47/10; A61K 47/26

(52) U.S. Cl. CPC A61K 31/454; A61K 9/0053; A61K 9/08; A61K 47/10; A61K 47/26

(57) ABSTRACT

Oral pharmaceutical solution comprising lenalidomide or a pharmaceutically acceptable salt thereof as active ingredient and a pharmaceutically acceptable carrier comprising water and a cosolvent selected from a glycol, or a polyol, wherein the pH of the solution is from 1.0 to 3.0.

### TECHNICAL FIELD

The present invention relates to oral pharmaceutical solutions comprising lenalidomide or a pharmaceutically acceptable lenalidomide salt.

### BACKGROUND OF THE INVENTION

Lenalidomide is an analog of thalidomide with antineoplastic activity. Lenalidomide inhibits TNF-alpha production, stimulates T cells, reduces serum levels of the cytokines vascular endothelial growth factor (VEGF) and basic fibroblast growth factor (bFGF), and inhibits angiogenesis. It also promotes G1 cell cycle arrest and apoptosis of malignant cells.

Lenalidomide is a racemic mixture, which has the chemical name 1-oxo-(2,6-dioxopiperidin-3-yl)-4-aminoisoindoline and the following structural formula:

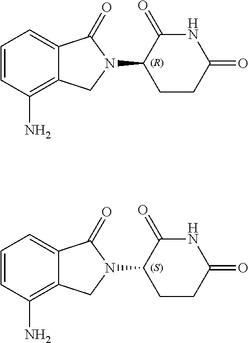

Lenalidomide and its process of manufacture were first disclosed in U.S. Pat. No. 5,635,517.

Lenalidomide is soluble in organic solvent/water mixtures and buffered aqueous solutions and exhibits the highest solubility of 18.0 mg/ml in a 0.1 N HCl aqueous solution (i.e. an aqueous solution with pH values around 1). Solubility in basic solutions decreases to about 0.4 to 0.5 mg/ml.

Besides pH, lenalidomide solubility is influenced by its crystalline form. As disclosed in U.S. Pat. No. 7,465,800, polymorph A is characterized as the most soluble crystalline form with a solubility of 6.2 mg/ml in an aqueous HCl solution at pH 1.8, containing 1% sodium lauryl sulfate. Polymorphs B and E exhibit solubility of 5.8 mg/ml and 4.7 mg/ml, respectively, under the same experimental conditions.

Currently, lenalidomide is approved in Europe as monotherapy for the maintenance treatment of adult patients with newly diagnosed multiple myeloma who have undergone autologous stem cell transplantation or as combination therapy with dexamethasone, or bortezomib and dexamethasone, or melphalan and prednisone for the treatment of adult patients with previously untreated multiple myeloma who are not eligible for transplant. It is also indicated as monotherapy for the treatment of adult patients with transfusion-dependent anaemia due to low- or intermediate-1-risk myelodysplastic syndromes associated with an isolated deletion 5q cytogenetic abnormality when other therapeutic options are insufficient or inadequate. In addition, it is indicated for the treatment of adult patients with relapsed or refractory mantle cell lymphoma. Furthermore, lenalidomide in combination with rituximab (anti-CD20 antibody) is indicated for the treatment of adult patients with previously treated follicular lymphoma The commercial lenalidomide capsules are marketed under the trade names Revlimid® and Ladevina®. These capsules are marketed in strengths of 2.5 mg, 5 mg, 7.5 mg, 10 mg, 15 mg, 20 mg and 25 mg of lenalidomide free base.

Although oral solid dosage forms such as tablets and capsules are very popular mainly due to ease of management, for certain users (e.g. children and the elderly) these forms are not necessarily a convenient option, especially due to difficulty in swallowing these forms. This lack of convenience results in high incidence of non-compliance and ineffective therapy.

Moreover, the Patient Information Leaflet (PIL) of Revlimid® tablets discloses a dosing scheme according to which the dosage is adjusted based on the medical condition of the patient, response to treatment, and lab test results. The concept of individualised, tailored treatment sets as prerequisite pharmaceutical forms, that enable dose fractioning, such as oral solutions.

Hence, as a means of both ensuring flexibility in dose titration and advanced patient compliance, the development of oral liquid dosage forms of lenalidomide, which can allow optimal dose selection is definitely an existing need.

Patent application WO 1998/03502 discloses in Example 23 a 0.2% injection or infusion solution comprising lenalidomide, sodium chloride, phosphate buffer pH 7.4 and demineralized water.

However, the desire for the development of an oral liquid dosage form that comprises lenalidomide is complicated by the fact that lenalidomide and its pharmaceutically acceptable salts are unstable in liquid environment under acidic and basic hydrolytic conditions as well as under oxidative conditions.

The present invention overcomes the problems of the prior art and provides an oral pharmaceutical solution, comprising lenalidomide or a pharmaceutically acceptable salt of lenalidomide, which exhibits excellent stability and extended lifetime.

### SUMMARY OF THE INVENTION

The present invention is directed to an oral pharmaceutical solution comprising lenalidomide or a pharmaceutically acceptable salt thereof in association with a pharmaceutically acceptable aqueous carrier.

The oral pharmaceutical solution according to the invention comprises lenalidomide or a pharmaceutically acceptable salt thereof as active ingredient and a pharmaceutically acceptable carrier comprising water and a cosolvent selected from the group consisting of a glycol and a polyol, wherein the pH of the solution is from 1.0 to 3.0.

The oral pharmaceutical solution according to the present invention provides the best alternative over conventional capsule or film coated tablet dosage forms. Apart from achieving better patient compliance, the oral solution of the present invention offers unique advantages such as more reproducible bioavailability and an option of a flexible dosing regimen.

### DETAILED DESCRIPTION OF THE INVENTION

The present invention provides an oral pharmaceutical solution comprising lenalidomide or a pharmaceutically acceptable lenalidomide salt as active ingredient.

Numerous combinations of cosolvents and surfactants such as polysorbate 20, 40, 60 or 80, povidone K25, K30, K29/32, 90F, sodium lauryl sulfate, hydroxypropyl cellulose, hydroxypropyl methylcellulose, poloxamer 20, 40, 60, fructose, amino acids and glucose, were evaluated by the present inventors but did not provide the desired solubility of lenalidomide and/or stability of the oral solution.

A composition was prepared according to example 23 of WO 1998/03502. The solution contained 5.0 g lenalidomide, 22.5 g sodium chloride, 300.0 g phosphate buffer with pH 7.4 and demineralized water. The pH of the final composition was 7.4.

However, precipitation was observed within minutes of the preparation of the composition.

Similar results (i.e. precipitation) were obtained a few minutes after lenalidomide was dissolved in an aqueous buffer at pH between 1.0 and 3.5.

Likewise, attempts to prepare an oral solution by combining lenalidomide with any one of arginine, leucine, povidone K25, sodium lauryl sulfate, hydroxypropyl methylcellulose, poloxamer 20, in an aqueous buffer at pH 2.0 or pH 2.5 led to the formation of precipitate within minutes of the preparation of the compositions.

After intensive testing it was surprisingly found that only when at least one polyol or at least one glycol is added to a lenalidomide solution, wherein the pH has been adjusted at low acidic values, it is possible to manufacture an oral solution instead of a suspension of lenalidomide or a salt thereof having excellent physicochemical stability.

The oral pharmaceutical solution according to the invention comprises lenalidomide or a pharmaceutically acceptable salt thereof as active ingredient and a pharmaceutically acceptable carrier comprising water and a cosolvent selected from the group consisting of a glycol and a polyol, wherein the pH of the solution is from 1.0 to 3.0.

As used throughout this description and claims, a “glycol” may be propylene glycol, polyethylene glycol or any other pharmaceutically acceptable polyalkylene glycol product such as those known in the art as the “PEG” series, or mixtures thereof.

Preferably the glycol is selected from propylene glycol, polyethylene glycol having an average molecular weight from 150 to 1500, or mixtures thereof.

More preferably, the polyethylene glycol has an average molecular weight of 200 (also known as polyethylene glycol 200, or PEG 200), or of 300 (also known as polyethylene glycol 300, or PEG 300), or of 400 (also known as polyethylene glycol 400, or PEG 400), or of 600 (also known as polyethylene glycol 600, or PEG 600), or of 800 (also known as polyethylene glycol 800, or PEG 800) or of 1450 (also known as polyethylene glycol 1450, or PEG 1450). Even more preferably, the polyethylene glycol has an average molecular weight of 400.

As used throughout this description and claims, the term “polyol” (polyhydric alcohol) refers to pharmaceutical excipients containing multiple hydroxyl groups. Although the term “polyol” includes sugar alcohols, it does not include sugars, i.e. carbohydrates, such as sucrose, glucose, dextrose, fructose and galactose. Typical examples of suitable polyols according to the invention are sugar alcohols such as, maltitol, glycerol, mannitol, sorbitol, xylitol, erythritol, isomalt and lactitol, as well as polyvinyl alcohol.

Preferably, the polyol is selected from glycerol, sorbitol, mannitol, maltitol, xylitol, erythritol, isomalt, lactitol, polyvinyl alcohol, or mixtures thereof.

More preferably, the polyol is selected from glycerol, sorbitol, mannitol, maltitol, xylitol, or mixtures thereof. Even more preferably, the polyol is glycerol.

Any pharmaceutically acceptable system which acts as a buffer in the pH region of the invention can be used in the oral pharmaceutical solution according to the invention. Examples of buffering agents include but are not limited to ascorbic acid, acetic acid, tartaric acid, citric acid monohydrate, sodium citrate, potassium citrate, acetic acid, sodium acetate, sodium hydrogen phosphate, sodium dihydrogen phosphate, calcium hydrogen phosphate, calcium dihydrogen phosphate, or mixtures thereof.

The oral pharmaceutical solution according to the invention may comprise lenalidomide or any pharmaceutically acceptable salt of lenalidomide such as hydrochloride, hydrobromide, methane sulfonate, ethane sulfonate, benzene sulfonate and p-toluenesulfonate. Preferably, the oral pharmaceutical solution according to the invention comprises lenalidomide.

Preferably, the oral pharmaceutical solution according to the invention comprises from 2 mg/ml to 10 mg/ml of lenalidomide or a pharmaceutically acceptable salt of lenalidomide. More preferably, the solution comprises from 4 mg/ml to 8 mg/ml of lenalidomide or a pharmaceutically acceptable salt of lenalidomide. Even more preferably, the solution comprises from 5 mg/ml to 7 mg/ml of lenalidomide or a pharmaceutically acceptable salt of lenalidomide.

Preferably, the total concentration of the cosolvent in the oral pharmaceutical solution according to the invention is from 100 to 800 mg/ml. More preferably, the total concentration of the cosolvent is from 150 mg/ml to 700 mg/ml. Even more preferably, the total concentration of the cosolvent is from 200 mg/ml to 600 mg/ml.

The term “mg/ml” used in the present description and claims means mg of the active ingredient or of the excipient(s) per 1 ml of the oral pharmaceutical solution.

Preferably, the pH of the solution is from 1.5 to 2.5.

According to a preferred embodiment, the oral pharmaceutical solution according to the invention comprises from 2 mg/ml to 10 mg/ml of lenalidomide or a pharmaceutically acceptable salt thereof, and a pharmaceutically acceptable carrier comprising water and a cosolvent selected from
a) a glycol selected from propylene glycol or polyethylene glycol having an average molecular weight from 150 to 1500, or
b) a polyol selected from glycerol, sorbitol, mannitol, maltitol, xylitol, erythritol, isomalt, lactitol, polyvinyl alcohol, or mixtures thereof,
wherein the total concentration of the cosolvent is from 100 mg/ml to 800 mg/ml, and
wherein the pH of the solution is from 1.0 to 3.0.

According to another preferred embodiment, the oral pharmaceutical solution according to the invention comprises from 2 mg/ml to 10 mg/ml of lenalidomide or a pharmaceutically acceptable salt thereof, and a pharmaceutically acceptable carrier comprising water and a cosolvent selected from
a) a glycol selected from propylene glycol or polyethylene glycol having an average molecular weight from 150 to 1500, or
b) a polyol selected from glycerol, sorbitol, mannitol, maltitol, xylitol, erythritol, isomalt, lactitol, polyvinyl alcohol, or mixtures thereof,
wherein the total concentration of the cosolvent is from 150 mg/ml to 700 mg/ml, and
wherein the pH of the solution is from 1.0 to 3.0.

According to another preferred embodiment, the oral pharmaceutical solution according to the invention comprises from 2 mg/ml to 10 mg/ml of lenalidomide or a pharmaceutically acceptable salt thereof, and a pharmaceutically acceptable carrier comprising water and a cosolvent selected from
a) a glycol selected from propylene glycol or polyethylene glycol having an average molecular weight from 150 to 1500, or
b) a polyol selected from glycerol, sorbitol, mannitol, maltitol, xylitol, erythritol, isomalt, lactitol, polyvinyl alcohol, or mixtures thereof,
wherein the total concentration of the cosolvent is from 200 mg/ml to 600 mg/ml, and
wherein the pH of the solution is from 1.0 to 3.0.

Preferably, the oral pharmaceutical solution according to the invention is free of surfactants.

The oral pharmaceutical solution according to the invention may also optionally contain additional excipients commonly used in preparing oral liquid compositions, such as an antimicrobial preservative, an antioxidant, a viscosity adjusting agent, a flavouring agent or a sweetener.

Examples of antimicrobial preservatives as referred to herein include but are not limited to sodium benzoate, benzoic acid, boric acid, sorbic acid and salts thereof, benzyl alcohol, parahydroxy benzoic acids and their alkyl esters, methyl, ethyl and propyl parahydroxy benzoates or their salts, or mixtures thereof.

Examples of antioxidants as referred to herein include but are not limited to sodium metabisulfite, butylated hydroxyanisole, butylated hydroxytoluene, ethylenediamine tetraacetic acid, ascorbic acid, α-tocopherol, propyl gallate, or mixtures thereof.

Examples of flavouring agents as referred to herein include but are not limited to fruit flavours such as orange, banana, strawberry, cherry, wild cherry, lemon and the like and other flavourings, such as cardamom, anise, mint, menthol, vanillin, bubble gum, or mixtures thereof.

Examples of sweeteners as referred to herein include but are not limited to sugars, i.e. carbohydrates, such as sucrose, glucose, dextrose, lactose, fructose and galactose as well as other sweetening agents known in the art such as sucralose, aspartame, acesulfame-K, thaumatin, mogroside, saccharin and salts thereof, sodium cyclamate, erythritol, glycyrrhizin, monosodium glycyrrhizinate, monoamonium glycyrrhizinate, or mixtures thereof.

The oral pharmaceutical solution according to the invention is preferably supplied as a multidose preparation. Each dose from a multidose container, such as an amber type III glass 50 or 100 ml bottle sealed with a suitable seal, such as a child resistant, tamper evident screw cap, can be administered by means of a device suitable for measuring the prescribed volume. The device is usually a spoon or a cup for volumes of 5 ml or multiples thereof, or an oral syringe for other volumes.

The oral pharmaceutical solution according to the invention exhibits excellent stability when it is stored at a temperature of 4-8° C. Importantly, the stability is maintained even when the storage temperature increases from 4-8° C. to 25° C. for several days.

The oral pharmaceutical solution of the present invention may be prepared using methods well known in the art and using regular manufacturing equipment.

For example, it may be prepared using the following process:

The active substance (lenalidomide or pharmaceutically acceptable salt of lenalidomide) and the excipients are weighed. Purified water is added to a vessel. The glycol(s) or polyol(s) are successively added to the vessel. The active substance is then added under continuous stirring. Hydrochloric acid solution (5N) is added under continuous stirring until the active substance is completely dissolved. An aqueous buffer solution, if present, is prepared in a separate vessel, and is added under continuous stirring until the active substance is completely dissolved. Preservative, if present, is also added under continuous stirring until complete dissolution. Flavor and the remaining excipients, if present, are successively added under continuous stirring, until complete dissolution. The pH of the solution is adjusted to the desired value by adding, for example, aqueous sodium hydroxide or hydrochloric acid. Finally, the volume is adjusted with purified water or with the buffer solution.

### EXAMPLES

The following examples show the influence of the proposed, according to the invention, aqueous carrier, on the solubility and stability of lenalidomide.

### Example 1

Purified water was added to a vessel. Glycol(s) or polyol was added to the vessel. Lenalidomide was then added under continuous stirring. An aqueous buffer solution (citric acid monohydrate/sodium citrate dihydrate/hydrochloric acid), was prepared in a different vessel and was added under continuous stirring until lenalidomide was completely dissolved. EDTA (if present) Sucralose and the flavor were also added under continuous stirring until complete dissolution. The pH of the solution was adjusted to the desired value by adding aqueous sodium hydroxide (1 N) or hydrochloric acid solution (5N). Finally, the volume was adjusted with purified water. The compositions were filled in amber type III glass 50 ml bottles.

The compositions of the prepared oral solutions are shown in Tables 1 & 2.

TABLE 1

| | mg/ml | | | |
|---|---|---|---|---|
| Component | Trial 1 | Trial 2 | Trial 3 | Trial I |
| Lenalidomide | 6 | 6 | 6 | 6 |
| Sucralose | 5 | 5 | 5 | 5 |
| PG | — | 250 | — | — |
| Glycerol | — | — | 250 | 350 |
| Orange flavor | 1.0 | 1.0 | 1.0 | 1.0 |
| Buffer | Qs | | | |
| (citric acid monohydrate/ sodium | | | | |
| citrate dihydrate/hydrochloric acid) | | | | |
| NαOH (1N) / HCL (5N) QS to | pH 2.2 | pH 2.2 | pH 2.2 | pH 2.2 |

| | |
|---|---|
| Purified Water | QS to 1 mL |

PG; Propylene glycol

TABLE 2

| | mg/ml | | | |
|---|---|---|---|---|
| Component | Trial 4 | Trial 5 | Trial 6 | Trial II |
| Lenalidomide | 6 | 6 | 6 | 6 |
| Sucralose | 5 | 5 | 5 | 5 |
| PG | — | 150 | 200 | 350 |
| PEG-400 | — | 50 | 50 | — |
| EDTA | 1.0 | 1.0 | 1.0 | 1.0 |
| Orange flavor | 1.0 | 1.0 | 1.0 | 1.0 |
| Buffer | Qs | | | |
| (citric acid monohydrate/ sodium | | | | |
| citrate dihydrate/hydrochloric acid) | | | | |
| NαOH (1N) / HCL (5N) QS to | pH 2.0 | pH 2.2 | pH 2.2 | pH 2.2 |

| | |
|---|---|
| Purified Water | QS to 1 mL |

PG; Propylene glycol,
PEG; Polyethylene glycol

The compositions were stored at 4-8° C. for a period of 180 days and at 25° C. for 5 days (samples were left on the lab bench for one day every 30 days of storage period). Quantification of lenalidomide and its impurities, in the prepared solution, was performed by HPLC. The results are shown in Tables 3 & 4.

Surprisingly, precipitation was observed within about two months after the preparation of the compositions of trials 1 & 4 (60 storage days in the refrigerator at (4-8° C.) plus 2 storage days at 25° C.).

TABLE 3

| | | | | | 180 days (4-8° C.)/ | | |
|---|---|---|---|---|---|---|---|
| | | T = 0 DAYS | | | 5 days (25° C.) | | |
| | | TRIAL | TRIAL | TRIAL | TRIAL | TRIAL | TRIAL |
| | | 2 | 3 | I | 2 | 3 | I |
| Appearance | | Clear colorless solution | | | Clear colorless solution | | |
| pH | | 2.2 | 2.2 | 2.2 | 2.2 | 2.2 | 2.2 |
| Assay (%) | | 100.4 | 99.7 | 100.6 | 99.2 | 98.6 | 99.9 |
| Impurities | LENA-01 | 0.01 | 0.03 | 0.03 | 0.1 | 0.1 | 0.04 |
| (%) | LENA-02 | ND | 0.01 | 0.01 | 0.1 | 0.1 | 0.01 |
| | RRT = 0.33 | 0.01 | 0.01 | 0.01 | 0.1 | 0.2 | 0.1 |
| | RRT = 0.70 | 0.03 | 0.04 | 0.02 | 0.1 | 0.1 | 0.1 |
| | RRT = 1.61 | 0.01 | 0.01 | 0.01 | 0.1 | 0.1 | 0.1 |
| | RRT = 1.76 | 0.01 | 0.01 | 0.01 | 0.2 | 0.2 | 0.1 |
| | RRT = 1.85 | ND | ND | ND | 0.1 | 0.1 | 0.1 |

| | | | | | | |
|---|---|---|---|---|---|---|
| Total impurities | 0.2 | 0.2 | 0.2 | 1.0 | 0.9 | 0.6 |
| (%) | | | | | | |

ND: Not Detected; RRT: Relative Retention Time
LENA-01: N-(2-(2,6-dioxopiperidin-3-yl)-1oxoisoindoin-4-yl)formamide
LENA-02: 3-(-4-nitro-1-oxoisoindolin-2-yl)piperidine-2,6-dione

TABLE 4

| | | | | | T = 180 days (4-8° C.)/ | | |
|---|---|---|---|---|---|---|---|
| | | T = 0 DAYS | | | 5 days (25° C.) | | |
| | | TRIAL | TRIAL | TRIAL | TRIAL | TRIAL | TRIAL |
| | | 5 | 6 | II | 5 | 6 | II |
| Appearance | | Clear colorless solution | | | Clear colorless solution | | |
| pH | | 2.2 | 2.2 | 2.2 | 2.2 | 2.2 | 2.2 |
| Assay (%) | | 99.6 | 98.9 | 100.1 | 98.2 | 97.6 | 99.7 |
| Impurities | LENA-01 | 0.01 | 0.02 | 0.03 | 0.1 | 0.1 | 0.02 |
| (%) | LENA-02 | ND | 0.01 | 0.02 | 0.1 | 0.1 | 0.01 |
| | RRT = 0.33 | 0.01 | 0.02 | 0.01 | 0.1 | 0.2 | 0.1 |
| | RRT = 0.70 | 0.03 | 0.03 | 0.04 | 0.2 | 0.2 | 0.1 |
| | RRT = 1.61 | 0.01 | 0.01 | 0.01 | 0.1 | 0.1 | 0.1 |
| | RRT = 1.76 | 0.01 | 0.02 | 0.01 | 0.1 | 0.1 | 0.1 |
| | RRT = 1.85 | ND | ND | ND | 0.2 | 0.3 | 0.1 |

| | | | | | | |
|---|---|---|---|---|---|---|
| Total impurities | 0.2 | 0.2 | 0.2 | 1.2 | 1.4 | 0.6 |
| (%) | | | | | | |

ND: Not Detected; RRT: Relative Retention Time
LENA-01: N-(2-(2,6-dioxopiperidin-3-yl)-1oxoisoindoin-4-yl)formamide
LENA-02: 3-(-4-nitro-1-oxoisoindolin-2-yl)piperidine-2,6-dione

The above results show that the solutions of trials 2, 3, 5, 6, I and II, containing at least one polyol or at least one glycol as cosolvent are stable enough for storage in a refrigerator, including temperature excursions at 25° C.

### Example 2

Trials III, IV, V, VI (compositions comprising buffer); Purified water was added to a vessel. The cosolvent (glycol(s) or polyol(s)) was added to the vessel. Lenalidomide was then added under continuous stirring. An aqueous buffer solution (citric acid monohydrate/sodium citrate dihydrate/hydrochloric acid) was prepared in a different vessel and was added under continuous stirring until lenalidomide was completely dissolved. Sucralose and the flavor were also added under continuous stirring until complete dissolution. The pH of the solution was adjusted to the desired value by adding aqueous sodium hydroxide (1 N) or hydrochloric acid solution (5N). Finally, the volume was adjusted with purified water. The compositions were filled in amber type III glass 50 ml bottles.

Trials VII, VIII, IX, X (compositions without buffer); Purified water was added to a vessel. The glycols and polyols were successively added to the vessel. Lenalidomide was then added under continuous stirring. Hydrochloric acid solution (5N) was added under continuous stirring until lenalidomide was completely dissolved. Sucralose and the flavor were also added under continuous stirring until complete dissolution. The pH of the solution was adjusted by adding aqueous sodium hydroxide (1N) or hydrochloric acid solution (5N) to the desired value. Finally, the volume was adjusted with purified water. The compositions were filled in amber type III glass 50 ml bottles.

The compositions of the prepared oral solutions are shown in Tables 5 & 6.

TABLE 5

| | mg/ml | | | |
|---|---|---|---|---|
| Component | Trial III | Trial IV | Trial V | Trial VI |
| Lenalidomide | 5 | 5 | 5 | 5 |
| Glycerol | 400 | 100 | — | — |
| Sorbitol | — | 300 | — | — |
| Maltitol | — | — | 400 | 300 |
| Mannitol | — | — | — | 100 |
| Sucralose | 5 | 5 | 5 | 5 |
| Orange flavor | 2.0 | 2.0 | 2.0 | 2.0 |
| Buffer | Qs | | | |
| (citric acid monohydrate/ sodium | | | | |
| citrate dihydrate/hydrochloric acid) | | | | |
| NαOH (1N) / HCL (5N) QS to | pH 2.2 | pH 2.2 | pH 2.2 | pH 2.2 |

| | |
|---|---|
| Purified Water | QS to 1 mL |

TABLE 6

| | mg/ml | | | |
|---|---|---|---|---|
| Component | Trial VII | Trial VIII | Trial IX | Trial X |
| Lenalidomide | 5 | 5 | 5 | 5 |
| Sucralose | 5 | 5 | 5 | 5 |
| Propylene glycol | 150 | 500 | — | 200 |
| PEG-400 | 100 | — | 400 | 200 |
| PEG-1450 | 50 | — | 50 | 100 |
| Orange flavor | 2.0 | 2.0 | 2.0 | 2.0 |
| HCL (5N) | Qs | | | |
| NαOH (1N) / HCL | pH 2.2 | pH 2.2 | pH 2.2 | pH 2.2 |
| (5N) QS to | | | | |

| | |
|---|---|
| Purified Water | QS to 1 mL |

The compositions were stored at 4-8° C. for a period of 180 days and at 25° C. for 5 days (samples were left on the lab bench for one day every 30 days of storage period). Quantification of lenalidomide and its impurities, in the prepared solution, was performed by HPLC. The results are shown in Tables 7 & 8.

TABLE 7

| | | | | | | T = 180 days (4-8° C.)/ | | | |
|---|---|---|---|---|---|---|---|---|---|
| | | T = 0 DAYS | | | | 5 days (25° C.) | | | |
| | | TRIAL | TRIAL | TRIAL | TRIAL | TRIAL | TRIAL | TRIAL | TRIAL |
| | | III | IV | V | VI | III | IV | V | VI |
| Appearance | | Clear colorless solution | | | | Clear colorless solution | | | |
| pH | | 2.2 | 2.2 | 2.2 | 2.2 | 2.2 | 2.1 | 2.2 | 2.2 |
| Assay (%) | | 100.0 | 100.2 | 100.6 | 100.4 | 99.5 | 99.7 | 100.4 | 100.1 |
| Impurities | LENA-01 | 0.01 | 0.01 | 0.02 | 0.02 | 0.02 | 0.04 | 0.05 | 0.02 |
| (%) | LENA-02 | ND | ND | 0.02 | 0.01 | 0.03 | 0.02 | 0.01 | 0.01 |
| | RRT = 0.33 | 0.01 | 0.01 | 0.01 | 0.01 | 0.1 | 0.1 | 0.1 | 0.1 |
| | RRT = 0.70 | 0.02 | 0.03 | 0.03 | 0.01 | 0.05 | 0.04 | 0.04 | 0.1 |
| | RRT = 1.61 | 0.01 | 0.01 | 0.01 | 0.01 | 0.1 | 0.1 | 0.1 | 0.1 |
| | RRT = 1.76 | 0.01 | 0.01 | 0.01 | 0.01 | 0.1 | 0.1 | 0.1 | 0.1 |
| | RRT = 1.85 | ND | ND | ND | 0.01 | 0.1 | 0.1 | 0.1 | 0.1 |

| | | | | | | | | |
|---|---|---|---|---|---|---|---|---|
| Total impurities (%) | 0.1 | 0.1 | 0.2 | 0.2 | 0.6 | 0.6 | 0.6 | 0.6 |

ND: Not Detected; RRT: Relative Retention Time
LENA-01: N-(2-(2,6-dioxopiperidin-3-yl)-1oxoisoindoin-4-yl)formamide
LENA-02: 3-(-4-nitro-1-oxoisoindolin-2-yl)piperidine-2,6-dione

TABLE 8

| | | | | | | T = 180 days (4-8° C.)/ | | | |
|---|---|---|---|---|---|---|---|---|---|
| | | T = 0 DAYS | | | | 5 days (25° C.) | | | |
| | | TRIAL | TRIAL | TRIAL | TRIAL | TRIAL | TRIAL | TRIAL | TRIAL |
| | | VII | VIII | IX | X | VII | VIII | IX | X |
| Appearance | | Clear colorless solution | | | | Clear colorless solution | | | |
| pH | | 2.2 | 2.2 | 2.2 | 2.2 | 2.2 | 2.2 | 2.2 | 2.1 |
| Assay (%) | | 100.9 | 100.1 | 99.9 | 101.3 | 100.1 | 99.6 | 99.4 | 100.6 |
| Impurities | LENA-01 | 0.01 | 0.01 | 0.02 | 0.02 | 0.01 | 0.02 | 0.02 | 0.02 |
| (%) | LENA-02 | ND | ND | 0.02 | 0.01 | 0.02 | 0.02 | 0.03 | 0.04 |
| | RRT = 0.33 | 0.01 | 0.01 | 0.01 | 0.01 | 0.1 | 0.1 | 0.1 | 0.1 |
| | RRT = 0.70 | 0.02 | 0.03 | 0.03 | 0.01 | 0.04 | 0.04 | 0.03 | 0.1 |
| | RRT = 1.61 | 0.01 | 0.01 | 0.01 | 0.01 | 0.1 | 0.1 | 0.1 | 0.1 |
| | RRT = 1.76 | 0.01 | 0.01 | 0.01 | 0.01 | 0.1 | 0.1 | 0.1 | 0.1 |
| | RRT = 1.85 | ND | ND | ND | 0.01 | 0.1 | 0.1 | 0.1 | 0.1 |

| | | | | | | | | |
|---|---|---|---|---|---|---|---|---|
| Total impurities (%) | 0.1 | 0.1 | 0.2 | 0.2 | 0.6 | 0.6 | 0.6 | 0.7 |

ND: Not Detected; RRT: Relative Retention Time
LENA-01: N-(2-(2,6-dioxopiperidin-3-yl)-1oxoisoindoin-4-yl)formamide
LENA-02: 3-(-4-nitro-1-oxoisoindolin-2-yl)piperidine-2,6-dione

From this study, it is further corroborated that in the presence of at least one polyol or at least one glycol, the stability of lenalidomide is acceptable for storage in a refrigerator, including temperature excursions at 25° C.

1. An oral pharmaceutical solution comprising lenalidomide or a pharmaceutically acceptable salt thereof as active ingredient and a pharmaceutically acceptable carrier comprising water and a cosolvent selected from a glycol, or a polyol, wherein the pH of the solution is from 1.0 to 3.0.

2. The oral pharmaceutical solution according to claim 1, wherein the glycol is selected from glycerol, polyethylene glycol having an average molecular weight from 150 to 1500, or a mixture thereof.

3. The oral pharmaceutical solution according to claim 2, wherein the polyethylene glycol has an average molecular weight of 400.

4. The oral pharmaceutical solution according to claim 1, wherein the glycol is propylene glycol.

5. The oral pharmaceutical solution according to claim 1, wherein the polyol is selected from maltitol, glycerol, mannitol, sorbitol, xylitol, erythritol, isomalt, lactitol, polyvinyl alcohol, or a mixture thereof.

6. The oral pharmaceutical solution according to claim 1, wherein the polyol is selected from glycerol, sorbitol, mannitol, maltitol, xylitol, or a mixture thereof.

7. The oral pharmaceutical solution according to claim 1, wherein the polyol is glycerol.

8. The oral pharmaceutical solution according to claim 1, wherein the concentration of lenalidomide or the pharmaceutically acceptable lenalidomide salt in the solution is from 2 mg/ml to 10 mg/ml.

9. The oral pharmaceutical solution according to claim 1, wherein the total concentration of the cosolvent in the solution is from 100 mg/ml to 800 mg/ml.

10. The oral pharmaceutical solution according to claim 1, wherein the total concentration of the cosolvent in the solution is from 150 mg/ml to 700 mg/ml.

11. The oral pharmaceutical solution according to claim 1, wherein the total concentration of the cosolvent in the solution is from 200 mg/ml to 600 mg/ml.

12. The oral pharmaceutical solution according to claim 1, wherein the pH of the solution is from 1.5 to 2.5.

13. The oral pharmaceutical solution according to claim 1, wherein the active ingredient is lenalidomide.

14. The oral pharmaceutical solution according to claim 1, wherein the solution does not comprise a surfactant.
